(19) United States
(12) Patent Application Publication
(10) Pub. No.: US 2009/0005772 A1
(43) Pub. Date: Jan. 1, 2009

(54) Tissue treatment apparatus

(75) Inventor: Keith Penny, Wellesley, MA (US)

Correspondence Address: OLIFF & BERRIDGE, PLC, P.O. BOX 320850, ALEXANDRIA, VA, 22320-4850, US

(73) Assignee: RHYTEC LIMITED, HUNGERFORD (GB)

(21) Appl. No.: 12/213,640

(22) Filed: Jun. 23, 2008

Related U.S. Application Data
(60) Provisional application No. 60/929,469, filed on Jun. 28, 2007.

(51) Int. Cl. A61B 18/18

(52) U.S. Cl. 606/40

(57) ABSTRACT

In tissue treatment apparatus for skin resurfacing having a handheld treatment instrument with electrodes connected to a radio frequency generator, a plasma jet is produced by ionising gas passing through a gas conduit in the instrument, the plasma emerging from a nozzle at an end of the conduit. Incorporated in the instrument is an optical detector that receives radiation emitted by the plasma directly from within the conduit and produces output signals which are processed to indicate a fault condition in the absence of radiation within a predetermined interval after the commencement of delivery of RF energy to the instrument or if the radiation is not of an approximately constant level during the delivery of RF energy.

### FIELD OF THE INVENTION

This invention relates to tissue treatment apparatus including a radio frequency (r.f.) generator and a treatment instrument connectible to the generator and to a source of ionisable gas for producing a plasma jet. The primary use of the system is skin resurfacing.

### BACKGROUND OF THE INVENTION

A tissue treatment system is disclosed in U.S. Pat. No. 6,723,091 filed Feb. 22, 2001 and U.S. Pat. No. 6,629,974 filed Feb. 13, 2002, and U.S. patent application Ser. No. 10/727,765 filed Mar. 5, 2004.

The complete disclosure of each of these patents and the application is incorporated in this application by reference. In this known system, a handheld treatment instrument has a gas conduit terminating in a plasma exit nozzle. There is an electrode associated with the conduit, and this electrode is coupled to a separate r.f. power generator which is arranged to deliver r.f. power to the electrode for creating a plasma from gas fed through the conduit. The delivered radio frequency power is typically at UHF (Ultra High Frequency) radio frequencies (r.f.) in the region of 2.45 Ghz and the instrument includes a structure resonant in that frequency region in order to provide an electric field concentration in the conduit for striking the plasma upstream of the exit nozzle, the plasma forming a jet which emerges from the nozzle and which can be used to effect local heating of a tissue surface.

The clinical effect of a system that delivers pulsed energy to the tissue of a patient is dependent on the amount of energy delivered, more particularly the instantaneous power integrated over the time of activation.

If the system was to malfunction, causing, for example, the duration of the applied pulse to increase substantially, or causing the energy of the pulse to increase substantially, the tissue onto which the plasma is being directed may be irreparably damaged. Likewise, if the system were to malfunction causing the duration of the applied pulse to be significantly shorter or causing the energy of the pulse to decrease substantially then the tissue onto which the plasma is being directed may not be treated adequately for the intended purpose. It is, therefore, important to be able to confirm that the energy delivered by the system corresponds to the setting of the generator (which may be set by the user) and is within the specification of the system.

In a previous implementation, the generator receives reflected r.f. power from the handheld treatment instrument (hereinafter “handpiece”) during the plasma pulse. The average level of the reflected r.f. power is then used to determine whether the power generation is normal (i.e. a relatively low level of r.f. power is reflected), or whether there is a problem preventing or limiting plasma generation (such as a faulty exit nozzle) by checking whether (i) the reflected power level falls between lower and upper threshold levels or, (ii) in the case of a more serious problem, such as a disconnected r.f. power cable, the reflected power level is above the upper threshold.

Detection of the reflected r.f. signal requires differentiation of the reflected signal from the much larger emitted r.f. signal. In an existing system, this differentiation is achieved using a circulator. A circulator has three ports: a first (input) port to receive r.f. power from the r.f. power generator, a second port that is connected to the handpiece, and a third port to which reflected r.f. power from the handpiece is directed. Under optimal conditions no reflected power reaches the input port and only reflected power is coupled to the third port, and therefore independent measurements of the emitted and reflected r.f. powers can be achieved.

A second method of differentiating between the emitted and reflected r.f. power, is to use a directional coupler, which has first and second (input and output) connections, together with a third connection that provides a directional sample of a main signal flowing through the device. Such a device can, according to the orientation of its insertion into the power flow path, provide forward or reverse samples for measurement by external circuitry.

The circulator and directional coupler described above are both relatively expensive, and reflections occurring other than those associated with the generation of plasma at the handpiece can compromise performance. Such multiple reflections cannot readily be analysed, and hence they cannot be distinguished from the reflected r.f. power signal.

Additionally, the reflected r.f. power signal is not a true indicator of satisfactory plasma generation. It would be possible for a fault to occur whereby little reflected r.f. power is produced because the emitted r.f. power is radiated into the surrounding space, and/or is converted into heat within the cable or the handpiece. The system would determine this erroneously as a good condition, corresponding to plasma generation even though plasma is absent.

An aim of the present invention is to provide an improved means of confirming the generation of satisfactory plasma in a system for tissue resurfacing.

### SUMMARY OF THE INVENTION

The present invention provides tissue treatment apparatus comprising a radio frequency (r.f.) generator, a treatment instrument, and an optical analysis device. The instrument has a gas conduit that terminates in a plasma exit nozzle and is connectible to a source of ionisable gas, and, associated with the conduit, a pair of electrodes connectible to the generator and arranged to produce an electric field in the conduit when energized with an r.f voltage by the generator thereby to produce a plasma in ionisable gas flowing through the conduit when the instrument is supplied with the gas. The optical analysis device comprises: at least one optical detector arranged to receive, directly from within the conduit, radiation emitted by the plasma; a processor stage for processing output signals from the or each optical detector so as to compare a representation of the output signals with a reference representation, and to generate a fault signal in response to a predetermined comparison result, the fault signal being indicative of a fault in the apparatus; and a control stage for controlling the generation of r.f. energy by the generator in response to the fault signal.

Advantageously, the or each optical detector receives radiation through an aperture formed in the side of the treatment instrument.

In a preferred embodiment, the tissue treatment apparatus further comprises at least one optical fibre for directing radiation emitted by the plasma to the at least one optical detector.

Preferably, the processor stage is arranged to control the flow of ionisable gas supplied to the instrument.

Advantageously, the tissue treatment apparatus further comprises a user interface, as well as means for indicating a fault to a user via the user interface. Preferably, the control stage is arranged for preventing further plasma production if a particular fault signal requiring such prevention is received by the processor. Advantageously, the control stage is also arranged for allowing further plasma production if a particular fault signal not requiring prevention of plasma production is received by the processor stage.

In the preferred embodiment, the processor and control stages form part of the generator and the processor stage is arranged to generate a fault signal when an output signal from the optical analysis device is indicative of (a) a lack of radiation within the conduit within a predetermined interval after commencement of delivery of r.f. energy to the instrument by the generator, or (b) the radiation within the conduit not remaining at least approximately constant during generation of r.f. energy by the generator. Thus, once a treatment pulse commences, the output of the optical analysis device and, therefore, the plasma itself, is monitored for consistency until the treatment pulse is terminated. This may be achieved by comparing the output from the optical analysis device with upper and lower output thresholds. Typically, if the output does not remain within a predetermined range whilst r.f. energy is demanded from the generator, the generation of r.f. energy is terminated.

According to another aspect of the present invention, there is provided a method of controlling a tissue treatment system having an r.f. generator, a treatment instrument, and an optical analysis device, the instrument being connectible to the generator and to a source of ionisable gas and operable to produce a plasma jet at a nozzle of the instrument when supplied with the ionisable gas and energised by the generator. The method comprises the steps of supplying ionisable gas from the gas conduit; actuating the generator to apply a radio frequency (RF) voltage to a pair of electrodes associated with the conduit to produce an electric field in the conduit and thereby to produce a plasma in the ionisable gas flowing through the conduit; receiving, in at least one optical detector, radiation emitted by the plasma, the radiation being received directly from within the conduit; comparing a representation of signals outputted from the at least one optical detector with a reference representation; generating a fault signal in response to a predetermined comparison result the fault signal being indicative of a fault in the tissue treatment apparatus; and controlling the generation of r.f. energy by the generator in response to the fault signal.

Preferably, the method further comprises the step of indicating a fault to a user and more preferably, the fault is indicated to the user via a user interface.

Advantageously, the radiation is received by the at least one optical detector via at least one optical fibre.

In the preferred embodiment of this invention, the optical detector is sensitive to radiation in the visible spectrum. However, the invention encompasses systems using an optical detector wholly or primarily sensitive to electromagnetic waves outside the visible spectrum, particularly ultra-violet or infra-red radiation.

The invention will now be described in greater detail below by way of example, and with reference to the drawings.

### BRIEF DESCRIPTION OF THE DRAWINGS

In the drawings:

FIG. 1 is a general view of a tissue treatment system in accordance with the invention;

FIG. 2 is a cross-section of a handpiece of a first embodiment of the invention;

FIG. 3 is a cross-section of a handpiece of a second embodiment of the invention;

FIG. 4 is a cross-section of a handpiece of a third embodiment of the invention;

FIGS. 5A, 5B and 5C are cross-sections of handpieces representing variations of the handpieces of the first, second and third embodiments respectively;

FIG. 6 is a block diagram of a system in accordance with the invention; and

FIG. 7 is a flow diagram showing fault detection methods used in the system of FIG. 6.

### DETAILED DESCRIPTION OF THE PREFERRED EMBODIMENTS OF THE INVENTION

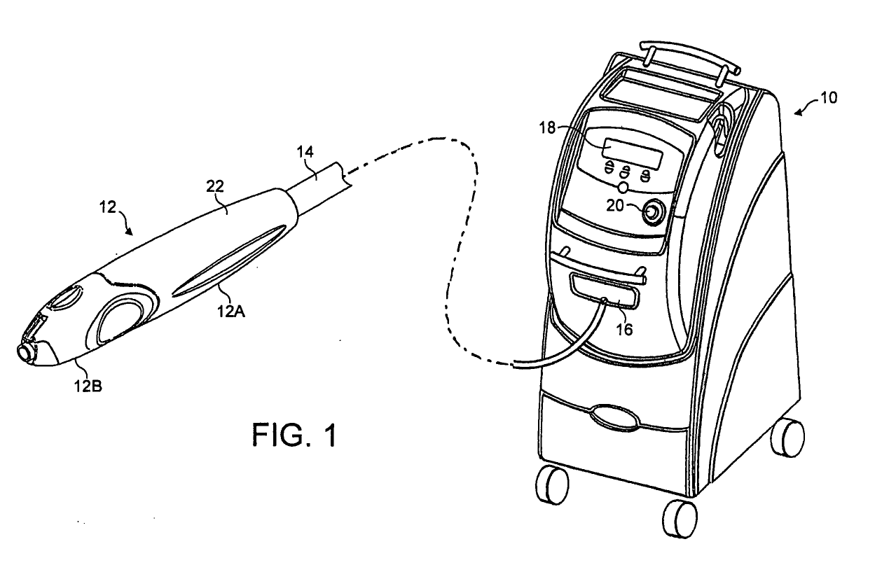

Referring to FIG. 1, a tissue treatment system has a base unit 10 and a handheld tissue treatment instrument 12, which is connected to the base unit by means of a cord 14. The instrument 12 comprises a handpiece having a re-usable handpiece body 12A and a disposable nose assembly 12B. The base unit 10 comprises a radio frequency (r.f.) generator 16, and a user interface 18 for setting the generator to different energy level settings.

The base unit 10 has an instrument holder 20 for storing the instrument when not in use.

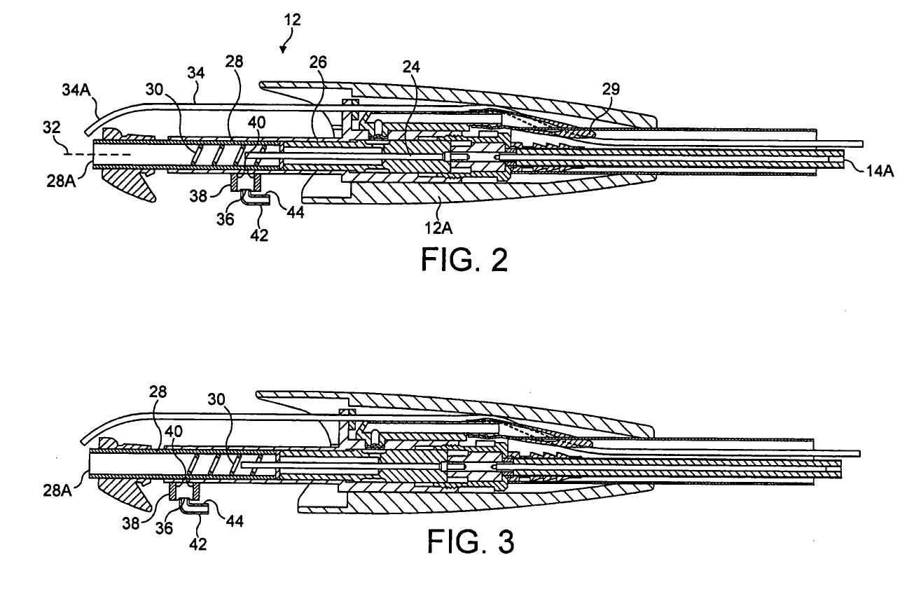

Within the cord 14 there is a coaxial cable for conveying r.f. energy from the generator 16 to the instrument 12, and a gas supply pipe for supplying nitrogen gas from a gas reservoir or source (not shown) inside the base unit 10. The core 14 also contains an optical fibre light guide 34 (see FIG. 2) for transmitting visible light to the instrument 12 from a light source in the base unit 10. At its distal end, the cord 14 passes into the casing 22 of the handpiece body 12A.

In the re-usable handpiece body 12A, the coaxial cable 14A is connected to inner and outer electrodes 24 and 26, as shown in FIG. 2, thereby coupling the electrodes to the generator 16 to receive r.f. power. The inner electrode 24 extends longitudinally within the outer electrode 26. Between them is a gas conduit in the form of a heat-resistant tube 28 (preferably made of quartz) housed in the disposable instrument nose assembly 12B (FIG. 1). When the nose assembly 12B is secured to the handpiece body 12A, the interior of the tube 28 is in communication with the gas supply pipe interior, the nose assembly 12B being received within the body 12A such that the inner and outer electrodes 24, 26 are associated with the tube, the inner electrode 24 extending axially into the tube and the outer electrode 26 extending around the outside of the tube.

A resonator in the form of a helically-wound stainless steel coil 30 is located within the quartz tube 28, the coil being positioned such that, when the disposable nose assembly 12B is secured in position on the handpiece body 12A, the proximal end of the coil is adjacent to the distal end of the inner electrode 24. The coil is wound such that it is adjacent to, and in intimate contact with, the inner surface of the quartz tube 28.

In use of the instrument, nitrogen gas is fed by a supply pipe 29 to the interior of the tube 28 where it reaches a location adjacent to the distal end of the inner electrode 24. When an r.f. voltage is supplied via the coaxial cable to the electrodes 24 and 26, an intense r.f. electric field is created inside the tube 28 in the region of the distal end of the inner electrode. The field strength is aided by the helical coil 30 which is resonant at the operating frequency of the generator and, in this way, conversion of the nitrogen gas into a plasma is promoted, the plasma exiting as a jet at a nozzle 28A of the quartz tube 28. The plasma jet, centred on a treatment beam axis 32 (this axis being the axis of the tube 28), is directed onto tissue to be treated, the nozzle 28A typically being held a few millimetres from the surface of the tissue.

The handpiece 12 also contains an optical fibre light guide 34 which extends through the cord 14 into the handpiece where its distal end portion 34A is bent inwardly towards the treatment axis defined by the quartz tube 28 to terminate at a distal end which defines an exit aperture adjacent the nozzle 28A. The inclination of the fibre light guide 34 at this point defines a projection axis for projecting a target marker onto the tissue surface.

Following repeated use of the instrument, the quartz tube 28 and its resonant coil 30 require replacement. The disposable nose assembly 12B containing these elements is easily attached and detached from the reusable part 12A of the instrument, the interface between the two components 12A, 12B of the instrument providing accurate location of the quartz tube 28 and the coil 30 with respect to the electrodes 24, 26.

In this first embodiment of the invention, an optical detector 36 is removably attached to an outer surface of the outer electrode 26 by means of a mounting member 38. The optical detector 36 is positioned such that it receives radiation from within the quartz tube 28 through a small aperture 40 in the surface of the outer electrode 26. The optical detector 36 is connected (a) to a power cable 42, the other end of which is connected to a power supply (not shown) to provide power to the optical detector, and (b) to a signal cable 44, the other end of which is connected to a central processing unit (CPU) (not shown) contained within the base unit 10. Any suitable optical detector 36 may be used, for example an integrated photo-optics sensor (model IPL 10530 DAL) made by Integrated Photo-Optics Limited. The aperture 40 is configured such that only a minimum amount of r.f. energy is leaked from within the quartz tube 28 whilst permitting adequate optical energy to reach the detector.

The aperture 40 is positioned such that the optical detector 36 detects radiation from the distal end of the inner electrode 24. During the early stages of plasma production, the region of the resonant coil 30 surrounding the distal end of the inner electrode 24 is responsible for forming arcs. The radiation emitted during the formation of these arcs is detected by the optical detector 36 and fed back to the CPU for analysis via the signal cable 44.

In a second embodiment of the invention, as illustrated in FIG. 3, the optical detector 36 is removably connected, as before, to the surface of the outer electrode 26 by means of a mounting member 38, but is positioned at a distal end of the resonant coil 30. Radiation emitted from within the resonant coil 30 passes through a small aperture 40 and is detected by the optical detector 36, the output of which is fed to the CPU via signal cable 44. In this embodiment, the optical detector 36 views plasma that is forming and flowing within the resonant coil 30 and from the distal end of the inner electrode, before it reaches the exist nozzle 28A of the quartz tube 28.

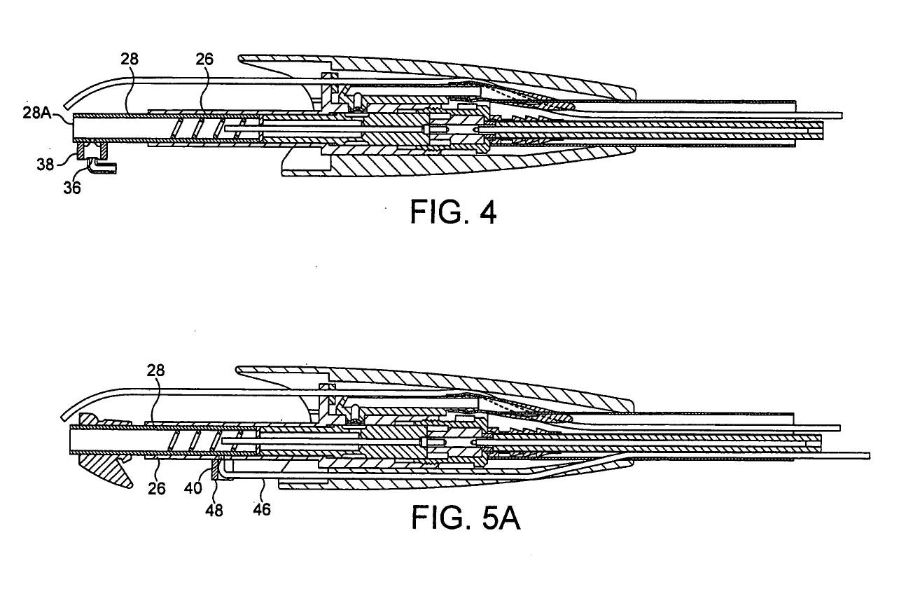

In a third embodiment of the present invention, as illustrated by FIG. 4, the optical detector 36 is removably connected to the exit nozzle 28A end of the quartz tube 28 by means of a mounting member 38. Since, in this embodiment, the optical detector 36 is positioned beyond the distal end of the outer conductor 26 and is attached directly to the quartz tube 28, which is substantially transparent, no aperture is required. As the plasma that is generated within the resonant coil 30 flows through the quartz tube 28, the quartz becomes hot. It is preferable, therefore, that the optical detector 36 is spaced from the surface of the quartz by means of a spacer (not shown), to avoid overheating, and possibly damaging, the optical detector.

In this embodiment, with the optical detector 36 positioned at the distal end of the quartz tube 28, the plasma radiation that is detected is substantially from the Lewis-Rayleigh afterglow. The quartz tube 28, and hence the mounting member 38, form part of the disposable assembly 12B so that, before disposing of the nose assembly, the optical detector 36 should first be removed from the mounting member, allowing it to be attached to the mounting member of a new nose assembly.

Alternatively, the optical detector may form an integral part of the disposable assembly with a releasable means of making the electrical connection to the generator.

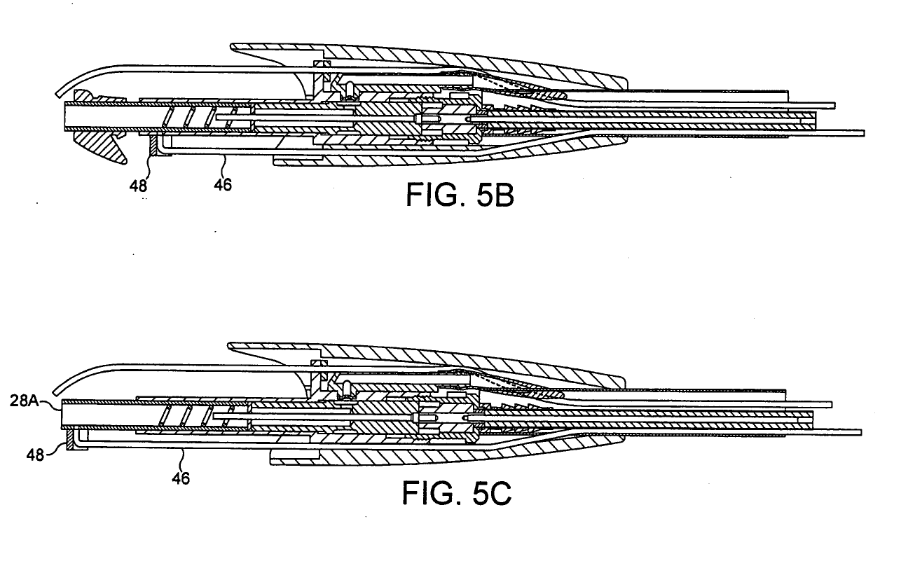

The embodiments shown in FIGS. 5A, 5B and 5C are variations of those shown in FIGS. 2, 3 and 4 respectively, whereby the optical detector 36 and the mounting member 38 are replaced by an optical fibre 46 removably attached to the outer electrode 26 or the outer surface of the quartz tube 28 respectively by means of an optical fibre mounting member 48. In the embodiments shown in FIGS. 5A and 5B, the optical fibre 46 receives radiation from within the quartz tube 28 through the small aperture 40 in the surface of the outer electrode 26. In the embodiment shown in FIG. 5C, the optical fibre 46 is positioned beyond the distal end of the outer electrode 26, and is attached directly to the substantially transparent quartz tube 28 adjacent the exit nozzle 28A. As in the embodiment of FIG. 4, in this case no aperture is required. The optical fibre 46 transmits the radiation to a detector (not shown) mounted in the base unit 10 or at another appropriate location.

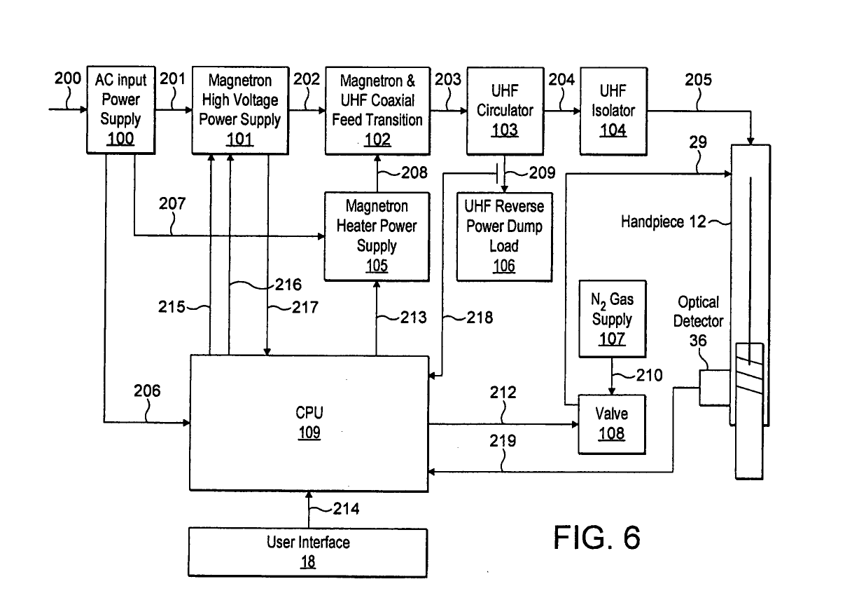

Reference is now made to FIG. 6, which is a block diagram of a system in accordance with the invention. An AC input power supply 100 receives external mains AC power 200, and generates voltages on supply lines 201, 206 and 207 to power circuits within a high voltage power supply 101 for a magnetron 102, a central processing unit (CPU) 109 and a magnetron heater power supply 105.

The magnetron 102 includes an associated coaxial feed transition, and receives a high voltage drive 202 from the magnetron high voltage power supply 101, and a low voltage, high current drive from the magnetron heater power supply 105, in order to generate r.f. power on an output line 203. In this embodiment, the r.f. power is generated in the UHF region, specifically at or near 2.45 Ghz R.f. power generated by the magnetron 102 is fed to a UHF circulator 103 the output of which on line 204 is fed to a UHF isolator 104, which provides an electrical isolation safety barrier. An output 205 of the isolator 104 is coupled to the handpiece 12 via the r.f. coaxial cable 14A (see FIG. 2) contained within cord 14.

Generation of the magnetron high voltage power supply voltage on line 202 requires two controls to be simultaneously present from the CPU 109. Firstly, a magnetron current demand control line 215 conveys a current demand signal from the CPU 109 to the magnetron high voltage power supply 101 to determine the instantaneous r.f. output power level of the r.f. power generated by the magnetron 102 on output 203 by determining the current level for the magnetron on the supply line 202. The generated current on line 202 is proportional to the voltage on the magnetron current demand control line 215. Since the .f. power level provided by the magnetron on output 203 is proportional to the supply current on supply line 202, the magnetron current demand signal on control line 215 determines the r.f. output power level. Secondly, an output enablement signal control line 216, which sends an enablement signal from the CPU 109 to the magnetron high voltage power supply 101, essentially turns the output of the high voltage power supply 101 on and off. Since the CPU 109 controls the enablement signal on control line 216, the duration of the output current 202 and, hence, the duration of the r.f. power output on line 203 are determined.

The CPU 109, therefore, sets the r.f. output power level by means of the magnetron current demand signal on line 215, and sets the duration of generation of the r.f. power output by means of the enablement signal on line 216.

Losses in r.f. power which occur in the UHF circulator 103, the isolator 104, their respective interconnections (not illustrated) and the coaxial cable 14A, which leads into the handpiece 12, are known or may otherwise be compensated for. The R.f. power level at the input 205 to the plasma generating handpiece 12 can, therefore, be determined.

A pressured nitrogen gas supply 107 is connected by connection means 210 to a gas valve 108, which is operated by the CPU 109 via a control feed 212. The nitrogen is fed via the gas supply pipe 29 (see also FIG. 2) into the handpiece 12.

During operation, the CPU 109 activates the control line 212, causing high pressure nitrogen gas to be fed to the handpiece 12. The magnetron current demand to the magnetron high voltage power supply 101 is set by means of a voltage level on the control line 215. When the gas from gas supply 107 is flowing into the handpiece 12, the enablement signal control line 216 is set, by the CPU 109 to cause generation of r.f. power 203 on magnetron output line at a power level according to the magnitude of the voltage on the magnetron current demand control line 215. The r.f. power on output line 203 is generated at a known power level for as long as the enablement signal on control line 216 activates the magnetron high voltage power supply 101.

Plasma generation typically begins within 0.5 ms of r.f. power being applied to the handpiece. Plasma generation ceases immediately when r.f. power is no longer being applied to the handpiece or when the r.f. power has fallen below a level required to sustain plasma generation.

During generation of an individual plasma pulse the following process take place:
1. Gas is released from the gas supply 107, according to a signal provided by the CPU 109 via the control line 212.
2. The r.f. power level of on power line 203, and, hence, the r.f. power supplied to the handpiece 12, is determined according to the voltage on the control line 215.
3. An individual pulse of a known power level P1 and pulse width T1 is generated by activation of the output 202 of the magnetron high voltage power supply 101 via the control line 216 for the same period T1 (ignoring propagation and other activation delays that are known and repeatable).
4. Plasma production typically begins within 0.5 ms of the start of the period T1.
5. At the end of the period T1 the control line 216 is disabled. In consequence the UHF r.f. power output 202 ceases and plasma generation also ceases.
6. The gas supply 107 is disabled by the CPU 109 via the valve control line 212 either prior to, or by the end of, period T1, or is maintained should another plasma pulse be required within a period T2 where T2 is a relatively short time, but otherwise is controlled as necessary to ensure efficient plasma production.

The user interface 18 is connected to the CPU 109 and provides means for a user to set required plasma pulse parameters.

The optical detector 36, connected to the outer surface of the handpiece 12 by means of a mounting member 38 (see FIGS. 2 to 4 and 5A to 5C), receives radiation from within the plasma generating chamber and feeds an output via adaptor output signal line 219 to the CPU 109. Analogue-to-digital conversion of the voltage on the signal line 219 takes place in the CPU 109. By this conversion, and by sampling the signal on signal line 219 at a sufficiently fast rate, the CPU 109 can determine the pulse optical output profile of the signal, and compare this with the expected behaviour for a normal plasma pulse throughout the duration of the individual pulse. If the output profile differs from a predetermined profile associated with a normal plasma pulse, the CPU 109 compares the profile with a number of predetermined error profiles, and can, therefore, determine a fault in plasma generation as it occurs. The user interface 18 may be used to indicate to the user the nature of the fault.

If, as a result of the fault occurring, immediate termination of the plasma generation is necessary, the CPU 109 disables the signal line 216, preventing further plasma generation.

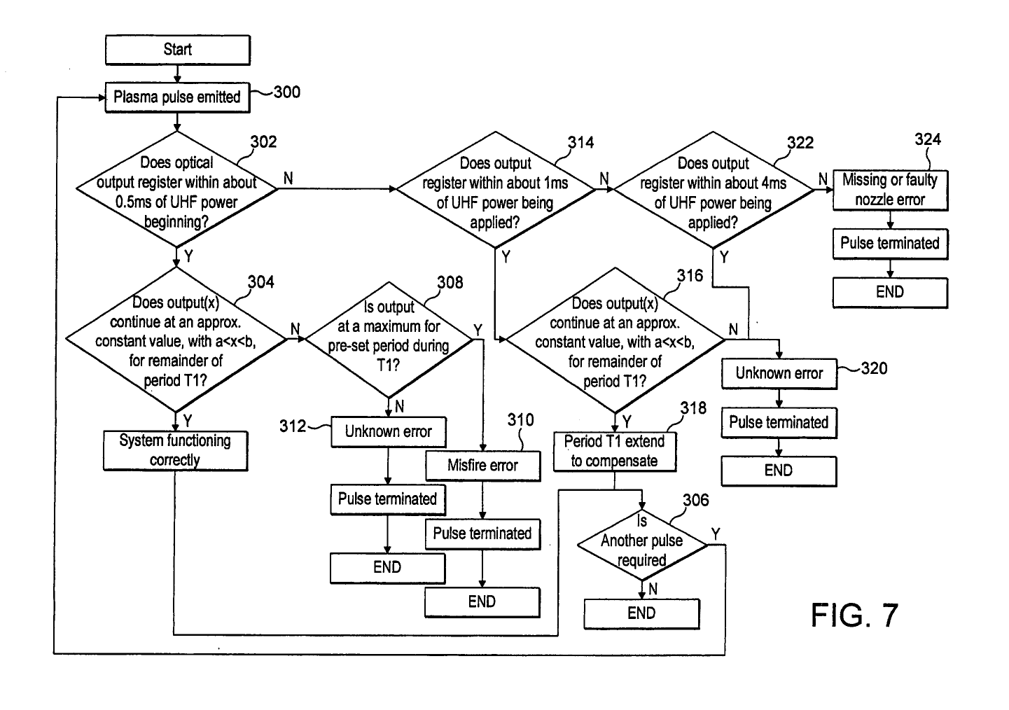

Referring now to FIG. 7, six possible errors may be determined by the CPU 109 in response to the output signal 219 received from the optical detector 36.

Once a plasma pulse has been emitted (step 300) the CPU 109 determines whether or not an optical output from the optical detector 36 is registered within approximately 0.5 ms of the r.f. power being applied (step 302). If so, the CPU 109 determines whether or not the output continues at an approximately constant value above a predetermined lower threshold value, a, and below a predetermined upper threshold value, b, until the supply of r.f. power is stopped (step. 304). If so, the system is deemed to be functioning correctly, with optimum plasma generation taking place. At the end of the period T1 the CPU disables the magnetron high voltage power supply 101 by means of a control signal on line 216, preventing further r.f. power generation.

If another pulse is required (determined by the parameters set by the user via the user interface 18) (step 306), the system returns to step 300, where another plasma pulse is emitted.

In a case where the output is registered within approximately 0.5 ms of the supply of r.f. power beginning, but where the output does not continue at an approximately constant value between the upper and lower threshold values b and a, the CPU 109 determines whether the output is at a maximum for a pre-set period, and, if so, registers a misfire error 310, the CPU 109 then prevents further r.f. power being supplied via signal line 216 for the remainder of the period T1 and informs the user via the user interface 18. If it is determined that, in step 308, the output is at a level below the lower threshold value a for a pre-set time, then the CPU 109 prevents further r.f. power being supplied via signal line 216 for the remainder of the period T1 and informs the user via the user interface 18.

If, after plasma pulse emission 300, an output signal is not registered within approximately 0.5 ms of the r.f. power generation beginning, the CPU 109 determines whether or not the output registers within approximately 1 ms of the r.f. power being applied (step 314). If an output is registered within approximately 1 ms, then the CPU 109 determines whether or not the output continues at an approximately constant value above the predetermined lower threshold value, a, and below the predetermined upper threshold value, b, If so, it is determined that a delayed plasma generation error 318 has occurred but is otherwise satisfactory. In this case the CPU may extend the period T1 in order to compensate for the delay in plasma generation as a means of ensuring accurate energy delivery. If, during step 316, it is determined that the output is above or below the upper and lower thresholds b and a respectively for a pre-set period then the CPU prevents further r.f. power being supplied via signal line 216 for the remainder of the period T1 and informs the user via the user interface 18 of an unknown error 320.

If, after the plasma pulse is emitted at step 300, an output is not registered after approximately 1 ms at step 314, the CPU 109 determines whether or not an output is registered within approximately 4 ms of the r.f. power being applied (step 322). If so, the CPU prevents further r.f. power being supplied, using control line 216, for the remainder of the period T1 and informs the user via the user interface 18 of an unknown error 320. If, however, an output is still not registered after approximately 4 ms of the r.f. power being applied (step 322), then the CPU 109 registers an error caused by a missing or faulty nozzle (step 324). In such a case, the CPU 109 prevents further r.f. power from being applied to the handpiece 12 via signal line 216.

It will be apparent to one skilled in the art that use of known techniques to attenuate, or to preferentially select parts of the optical spectrum (whether visible to the human eye or not), whether through use of optical filters or through the spectral response characteristics of the detector or detectors or a combination thereof, may be employed to optimise the ability of the system to determine a fault condition. It will also be apparent to one skilled in the art that use of a device such as a spectrometer may be employed in addition to or in place of a simpler optical detector. It will also be apparent to one skilled in the art, that a method such as selectively weighting the contribution to the overall optical level detected, according to different angles of entry of light into the detector and associated optical filters, may be employed.

1. Tissue treatment apparatus comprising a radio frequency (r.f.) generator, a treatment instrument, and an optical analysis device, wherein the instrument has a gas conduit that terminates in a plasma exit nozzle and is connectible to a source of ionisable gas, and, associated with the conduit, a pair of electrodes connectible to the generator and arranged to produce an electric field in the conduit when energized with a radio frequency (RF) voltage by the generator thereby to produce a plasma in ionisable gas flowing through the conduit when the instrument is supplied with the gas, and wherein the optical analysis device comprises:
at least one optical detector arranged to receive, directly from within the conduit, radiation emitted by the plasma;
a processor stage for processing output signals from the at least one optical detector so as to compare a representation of the output signals with a reference representation, and to generate a fault signal in response to a predetermined comparison result, the fault signal being indicative of a fault in the apparatus; and
a control stage for controlling the generation of r.f. energy by the generator in response to the fault signal.

2. Tissue treatment apparatus according to claim 1, wherein the at least one optical detector receives radiation through an aperture formed in the side of the treatment instrument.

3. Tissue treatment apparatus according to claim 1, further comprising at least one optical fibre for directing radiation emitted by the plasma to the at least one optical detector.

4. Tissue treatment apparatus according to claim 1, wherein the processor is arranged to control the flow of ionisable gas supplied to the instrument.

5. Tissue treatment apparatus according to claim 1, further comprising a user interface.

6. Tissue treatment apparatus according to claim 5, further comprising means for indicating a fault to a user via the user interface.

7. Tissue treatment apparatus according to claim 1, wherein the control means is arranged to prevent further plasma production if a particular fault signal requiring such prevention is received by the processor.

8. Tissue treatment apparatus according to claim 1, wherein the control means is arranged to allow further plasma production if a particular fault signal not requiring prevention of plasma production is received by the processor.

9. Tissue treatment apparatus according to claim 1, wherein the processor and control stages form part of the generator.

10. Tissue treatment apparatus according to claim 1, wherein the processor stage is arranged to generate a fault signal when an output signal from the optical analysis device is indicative of a lack of radiation within the conduit within a predetermined interval after commencement of delivery of r.f. energy to the instrument by the generator.

11. Tissue treatment apparatus according to claim 1, wherein the processor stage is arranged to generate a fault signal when an output signal from the optical analysis device is indicative of the radiation within the conduit not remaining approximately constant during generation of r.f. energy by the generator.

12. A method of controlling a tissue treatment apparatus having a radio frequency (r.f.) generator, a treatment instrument having a gas conduit that terminates in a plasma exit nozzle, and an optical analysis device, the instrument being connectible to the generator and to a source of ionisable gas and operable to produce a plasma jet at the nozzle of the instrument when supplied with the ionisable gas and energised by the generator, the method comprising the steps of:
supplying ionisable gas from the gas conduit;
actuating the generator to apply a radio frequency (RF) voltage to a pair of electrodes associated with the conduit to produce an electric field in the conduit and thereby to produce a plasma in the ionisable gas flowing through the conduit;
receiving, in at least one optical detector, radiation emitted by the plasma, the radiation being received directly from within the conduit;
comparing a representation of signals outputted from the at least one optical detector with a reference representation;
generating a fault signal in response to a predetermined comparison result the fault signal being indicative of a fault in the tissue treatment apparatus; and
controlling the generation of r.f. energy by the generator in response to the fault signal.

13. A method according to claim 12, wherein the fault signal is generated when the comparison step indicates a lack of radiation within the conduit within a predetermined time interval after commencing actuation of the generator.

14. A method according to claim 12, wherein the fault signal is generated when the comparison step indicates that the radiation within the conduit has not remained approximately constant during actuation of the generator.

15. A method according to claim 12, wherein the radiation is received by the at least one optical detector via at least one optical fibre.
